(19) United States
(12) Patent Application Publication
(10) Pub. No.: US 2011/0288465 A1
(43) Pub. Date: Nov. 24, 2011

(54) CANCER SURGERY AND TARGETED KILLING

(76) Inventors: Ming Zhao, San Diego, CA (US); Hiroyuki Kishimoto, San Diego, CA (US); Hiroaki Kimura, Kanazawa (JP)

(21) Appl. No.: 12/950,619

(22) Filed: Nov. 19, 2010

Related U.S. Application Data
(60) Provisional application No. 61/263,251, filed on Nov. 20, 2009.

(51) Int. Cl. A61B 18/18

(52) U.S. Cl. 604/20; 606/2

(57) ABSTRACT

Cancer treatment is improved by performing fluorescence-guided surgery and cell killing mediated by fluorescent proteins individually or together.

### CROSS-REFERENCE TO RELATED APPLICATION

This application claims priority from U.S. provisional application 61/263,251 filed 20 Nov. 2009. The contents of this document are incorporated herein by reference.

### FIELD OF THE INVENTION

The invention is directed to cancer treatments using fluorescent proteins. More specifically it includes both fluorescence-guided surgery and cell killing mediated by fluorescent proteins individually or together.

### BACKGROUND ART

Fluorescent proteins can be selectively replicated in malignant cells in the context of normal tissue by employing telomerase-specific viral vectors. These have been disclosed for therapeutic purposes and for use in in vivo imaging. See, for example, Umeoka, T., et al., Cancer Res. (2004) 64:6259-6265; Kishimoto, H., et al., Nat. Med. (2006) 12:1213-1219 among others. Specifically, one of these vectors, OBP-401 which expresses green fluorescent protein (GFP) and the viral E1 genes essential for replication under control of the human telomerase reverse transcriptase promoter has been used to visualize human colon tumors in in vivo models and to monitor metastases. The present inventors are believed the first to suggest using this selective fluorescent technique either to effect guidance for surgical removal of tumors or to effect direct killing of tumor cells in the context of normal tissue. Surgical guidance was described by applicants in Kishimoto, H., et al., PNAS (2009) 106:14514-14517 and targeted killing by Kimura, H., et al., J. Cellular Biochem. (2010) 110:1439-1446. The contents of these documents are also incorporated herein by reference.

### DISCLOSURE OF THE INVENTION

The invention takes advantage of the selective expression of fluorescent proteins in malignant cells to use the fluorescence generated as a guide for surgery and as a substrate for direct killing of the cells. In laboratory tumor models, fluorescent labeling can also be effected by other means, such as implantation of tumors already bearing label.

Thus, in one aspect, the invention is directed to a method to remove tumors surgically which method comprises employing selectively labeled cancer tissue, which has been modified to express a fluorescent protein, and, using fluorescence as a guide, removing only fluorescent tissue from a subject.

In another aspect, the invention is directed to a method selectively to kill tumor cells in the context of normal tissue which method comprises irradiating tissue containing fluorescently labeled tumor cells with ultraviolet light, especially UVC, wherein said tumor cells have been selectively labeled to express a fluorescent protein.

These techniques, especially when used in combination can result in a specific and complete removal of tumor tissue from a subject. In still another aspect, the invention is directed to a method to effect efficient removal of solid tumors from a subject which method employs fluorescence-guided surgery followed by irradiation with ultraviolet (UV) light, especially UVC.

### BRIEF DESCRIPTION OF THE DRAWINGS

FIG. 1 is a graph that shows the effect of UV irradiation on fluorescent dual-labeled tumor cell lines.

### MODES OF CARRYING OUT THE INVENTION

The availability of a method to label selectively tumor cells in the context of normal tissue offers an opportunity to enhance the efficacy of tumor treatment. Photodynamic therapy has been employed, for example, using porphyrin-related compounds as photosensitizing drugs to kill tumor cells in vivo. However, the selectivity of these drugs is not perfect. Surgical removal of tumors is also well known, but it is difficult to distinguish tumor tissue from normal tissue surrounding it. In both cases, the effects of the treatment are not focused, and thus often either not complete or overly aggressive.

In one embodiment of the invention, the exquisite selectivity of the labeling of tumor cells by virtue of selective viral replication in the intracellular generation of fluorescent protein is employed. This method of tumor labeling is especially useful when applied to indigenous tumors, as would be found in human or veterinary patients. However, the surgical and irradiation techniques of the invention can also be applied in the context of laboratory models where alternative methods of labeling the tumor selectively can be employed. For example, the tumor may be produced by implantation of fluorescently-labeled tumor cells. The surgical and radiation techniques can be applied to test effectiveness of treatments and surgical protocols in these models.

It is by now well established that multiple fluorescent proteins are commercially available in various colors. The cytotoxic effect of irradiation of cells containing these proteins is not a result of fluorescence, but rather some other change in the molecule, possibly the generation of singlet oxygen. For standard imaging techniques using these proteins, visible light may be used as the excitation source. However, in the techniques of the invention to effect cytotoxicity, ultraviolet light at a wavelength absorbed by the particular fluorescent protein in question is required.

For historical reasons, fluorescent proteins are often referred to as green fluorescent proteins or “GFP.” This is because the initially isolated fluorescent protein was, in fact, green. However, the fluorescent proteins useful in the invention may be of various colors, including red, yellow, and the like. If a specific non-green color is intended, this is usually noted—i.e., “RFP” for red fluorescent protein. However, the designation GFP may not necessarily refer to a protein that is green, but that is merely fluorescent.

The subjects for these treatments are any vertebrate subject, including mammals and humans. Veterinary patients may be the subjects of treatment. Animals that are laboratory models are also usefully employed to evaluate particular therapeutic and diagnostic methods.

In the case of laboratory animals, such as mice, rats, rabbits and the like, tumors may be labeled in a variety of ways, including labeling tumor cells before introducing them into the model. For any possible clinical use, however, the in vivo labeling technique such as that described in the examples below is required. The illustrated OBP-401 is not the only viral or other vector that could be so used, and others are known in the art.

It is particularly advantageous to use the guided surgery and cell killing techniques in combination. Thus, the bulk of a solid tumor is removed using the guidance of the fluorescing cells and the subject is then subjected to UV radiation in the appropriate areas to “mop-up” any cells might have been missed.

The following examples are intended to illustrate, but not to limit the invention.

### Preparation A

### Labeling Peritoneal Carcinomatosis with OBP-401

OBP-401 is described by Kishimoto, H., et al., Nat. Med. (2006) 12:1213-1219 and by Fujiwara, T., et al., Int. J. Cancer (2006) 119:432-440. OBP-401 is a modified adenovirus that contains a replication cassette with the human telomerase reverse transcriptase promoter driving expression of the viral E1 genes and the inserted GFP gene. Viral replication and GFP gene expression occur only in the presence of active telomerase—that is, in malignant tissue.

Peritoneal carcinomatosis was induced in the abdominal cavity of nude mice by inoculating 3×106 red fluorescent HCT-1,6-RFP human colorectal cancer cells. Various-sized peritoneal disseminated nodules developed within 12 days. These were clearly visible by fluorescence imaging using a long-pass filter and/or a specific RFP filter. Even very small disseminated nodules were illuminated by RFP fluorescence. Although there was some autofluorescence from adjacent organs visible, the tumor nodules were not visible through a GFP filter.

Once the malignant nodules were established at 12 days after intraperitoneal (i.p.) implantation of HCT-1,6-RFP cells, 1×108 PFU OBP-401 were injected into the mouse abdominal cavity. Selective color filters showed that the HCT-1,6-RFP disseminated nodules expressed GFP fluorescence as well as RFP when examined 5 days later. RFP fluorescence was essentially coincident with that of GFP. These results indicate that i.p. injection of OBP-401 efficiently infected and labeled disseminated cancer.

### Example 1

### Fluorescence-Guided Resection of Disseminated Peritoneal Tumors

Rather than RFP labeled cells, the peritoneal carcinomatosis model with nonfluorescent HCT-116 human colon cancer cells was used. Mice with peritoneal carcinomatosis were injected i.p. with OBP-401 at a dose of 1×108 PFU. Five days after viral administration, laparotomy was performed to remove intra-abdominal disease using fluorescence-guided navigation under anesthesia. Disseminated cancer nodules, which would otherwise be undetectable, were clearly visible by bright GFP fluorescence. The resected nodules were visualized as frozen sections under both fluorescence and after hematoxylin and eosin (H&E) staining. These results show that OBP-401-labeling can be used for guiding cytoreduction surgery of disseminated cancer.

### Example 2

### Selective Killing In Vitro

In this example, dual color cancer cells in which RFP is expressed in the cytoplasm and GFP in the nucleus were used. Preparation of such cells is described by Yamamoto, et al., Cancer Res. (2004) 64:4251-4256. Preparation of viral vectors is further described by Hoffman, R., et al., Nat. Protoc. (2006) 1:775-782; Nat. Protoc. (2006) 1:928-935; Nat. Protoc. (2006) 1:1429-1438. These documents are incorporated herein by reference. The cancer cell lines used were HT1080 human fibrosarcoma (HT1080), 143B human osteosarcoma (143B), Lewis lung carcinoma (LLC), and XPA-1 human pancreas cancer (XPA-1). Cells were maintained in RPMI 1640 supplemented with 10% fetal bovine serum, 1% penicillin and streptomycin at 37° C. in a humid atmosphere containing 5% CO₂. For in vivo studies, LLC-dual-color cells were used.

For UV irradiation, the cells were cultured on Lab-Tek™ II Chambered Coverglasses (Nalgen Nunc, Rochester, N.Y.) coated with BD™ fibronectin (BD Bioscience, Bedford, Mass.) at 5 μg/cm². The cells were irradiated with UV light from the bottom of the chamber using a Benchtop 3UV™ transilluminator (UVP®, LLC, Upland, Calif.), which emits UVC with an emission peak at 254 nm; UVB with an emission peak at 302 nm; and UVA with an emission peak at 365 nm. For in vivo UV irradiation, a customized UVC pen light (emission peak at 265 nm, UVP®) was used. The UV dose was measured with a UVX Radiometer (UVP®).

Cells were seeded into the glass chambers. After 48 h culture, the cells were irradiated with various doses UVA, UVB, or UVC (25-200 J/m²). Twenty-four hours after irradiation, the number of apoptotic and non-apoptotic cells was determined by nuclear structure under fluorescence microscopy. Eight chambers were used for each cell line and each UV dose.

Each of the four types of cancer cell lines (143B, HT1080, LLC, and XPA-1) were irradiated with 50 J/m² UVC or 100 J/m² UVB. Twenty-four hours after irradiation, the number of apoptotic and non-apoptotic cells were counted under fluorescence microscopy. Eight chambers were used for each cell line and every UV dose.

Imaging of UV-induced cancer-cell killing was started immediately after 143B dual-color cells were irradiated with 25 J/m² UVC. Real-time images were captured with the Olympus® OV100 Small Animal Imaging System (Olympus Corp., Tokyo, Japan) every 15 min for 12 h.

Twenty-four hours after UVA exposure, most cells were viable without change in morphology, even when the UVA dose was increased up to 200 J/m².

After UVB irradiation, apoptotic cells began to appear at 50 J/m². With 200 J/m² UVB, approximately 85% of the irradiated cells became apoptotic as determined by nuclear condensation and fragmentation. In a few cells, the cytoplasm was rounded without change in nuclear shape.

UVC irradiation induced cancer cells apoptosis at the highest frequency. As little as 25 J/m² UVC irradiation killed approximately 70% of 143B dual-color cells. The frequency of cell killing plateaued at 100 J/m². The morphological features of UVC irradiated cells were similar to those irradiated by UVB, in which cell shrinkage, nuclear condensation, and fragmentation occurred.

These results indicated that UV-induced cancer cell death was wavelength and dose dependent.

143B, HT1080, LLC, and XPA-1 dual-color cells were irradiated with 50 J/m² UVC or 100 J/m² UVB, based on the data described above. Twenty-five hours after 50 J/m² UVC irradiation, over 80% of the 143B cells became apoptotic, whereas less than 30% of the LLC cells became apoptotic. UVB irradiation was also more effective on 143B cells than on LLC cells. The other cell lines were intermediately sensitive to UVC and UVB. This result showed the sensitivity to UV irradiation was cell-line dependent.

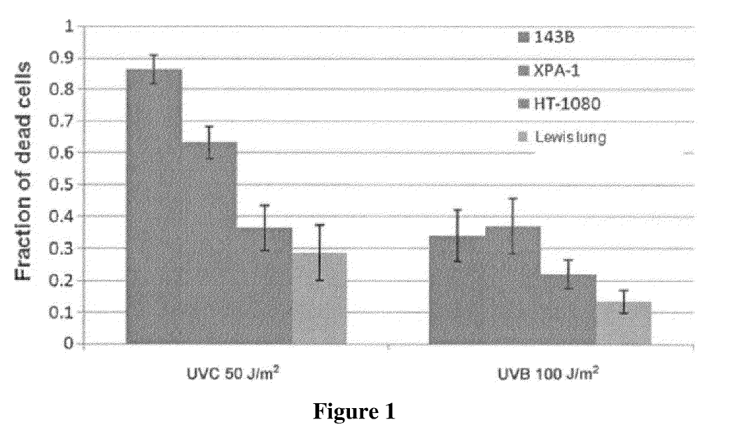

These results are illustrated graphically in FIG. 1.

### Example 3

### Selective Killing In Vivo

To model minimal residual cancer (MRC) after surgery, five athymic NCR nude (nu/nu) mice (Charles River Laboratories, Wilmington, Mass.) were first anesthetized with a ketamine mixture (10 μl ketamine HCL, 7.6 μl xylazine, 2.4 μl acepromazine maleate, and 10 μl H₂O s.c.) via s.c. injection, Rectangular incisions (each side 5 mm) were made bilaterally on the flanks in each mouse. LLC-dual-color cells (5×10⁵) in 10 μA PBS were then injected in the incised area. Incisions were closed with 6-0 sutures. Forty-eight hours after cancer cell injection, the bilateral incisions were reopened and UVC was irradiated only on the right side for 180 sec with the customized UV pen light. The estimated UVC exposure was 100 J/m². The mice were imaged once every 5 days after UVC exposure (days 5, 10, and 15), using the iBox® Scientia Small Animal Imaging System (UVP) to evaluate fluorescent tumor areas (mm²).

UVC irradiation suppressed tumor growth in all five treated mice. The average fluorescent area of LLC-dual-color tumors on the untreated flank was significantly larger than on the UVC irradiation side at days 5, 10, and 15. In one mouse, UVC irradiation completely inhibited tumor formation. No apparent side effects of UVC exposure were observed.

1. A method to remove tumors surgically from a subject which method comprises removing only fluorescent tissue from said subject, wherein the cancer tissue of said subject has been selectively modified to express a fluorescent protein.

2. A method selectively to kill tumor cells in the context of normal tissue of a subject which method comprises irradiating, with ultraviolet light, tissue of the subject containing tumor cells that have been selectively labeled to express a fluorescent protein.

3. A method to effect efficient removal of solid tumors from a subject which method comprises removing only fluorescent tissue from said subject, wherein the cancer tissue of said subject has been selectively modified to express a fluorescent protein, followed by irradiating with ultraviolet light tissue containing tumor cells that have been selectively labeled to express a fluorescent protein.

4. The method of claim 2 wherein the ultraviolet light is UVC.

5. The method of claim 3 wherein the ultraviolet light is UVC.

6. The method of claim 1 wherein the subject is human and wherein the cancer tissue has been selectively modified to express a fluorescent protein by administering a viral vector comprising an expression system for a protein essential for replication and a fluorescent protein under the control of a telomerase reverse transcriptase promoter.

7. The method of claim 2 wherein the subject is human and wherein the cancer tissue has been selectively modified to express a fluorescent protein by administering a viral vector comprising an expression system for a protein essential for replication and a fluorescent protein under the control of a telomerase reverse transcriptase promoter.

8. The method of claim 3 wherein the subject is human and wherein the cancer tissue has been selectively modified to express a fluorescent protein by administering a viral vector comprising an expression system for a protein essential for replication and a fluorescent protein under the control of a telomerase reverse transcriptase promoter.
